(19) United States
(12) Patent Application Publication
(10) Pub. No.: US 2010/0098789 A1
(43) Pub. Date: Apr. 22, 2010

(54) Compositions of ground/powdered nuts/nut butters with curcuminoids/turmeric/mix having improved health benefits and oxidative stability

(76) Inventors: Rema Balambika, Staten Island, NY (US); Krishnasawami Raja, Staten Island, NY (US)

Correspondence Address: Isaac A. Angres;Suite 304B, 2001 Jefferson Davis Highway, Arlington, VA, 22202, US

(21) Appl. No.: 12/588,296

(22) Filed: Oct. 9, 2009

Related U.S. Application Data
(60) Provisional application No. 61/196,651, filed on Oct. 20, 2008.

(51) Int. Cl. A61K 36/906

(52) U.S. Cl. 424/756

(57) ABSTRACT

A turmeric, turmeric components, curcumin or curcuminoid modified nut or seed butter spread known as A which is resistant to oxidation with improved cu/co/tc/tu/mx solubility and possessing some human health benefits comprising: 0.2-70% of components selected from the group consisting of curcumin, curcuminoids, turmeric components and turmeric; and 30-99.8% of nut or seed particles and/or nut/seed butters as a dispersing medium. This spread has increased resistance to oxidation and bacterial attack as compared to the nut or seed butter alone. Based on health studies of curcumin, the modified nut or seed butter spread is also expected to have significant preventative health benefits including effectiveness as a chemopreventive, anti-Alzheimer's, anti-inflammatory, and antibacterial agent. The nut/seed butter medium improves the solubility and bioavailability of cu/co/tc/tu/mx. The invention is taste perfected for the western palate, it will serve as a novel vehicle for making curcumin/turmeric consumption an integral part of the western diet.

This application claims the priority benefit under 35 U.S.C. section 119 of U.S. Provisional Patent Application No. 61/196,651 entitled “Spreads and Dips Incorporating turmeric/curcuminoids/curcumin and Other Natural Additives”, filed Oct. 20, 2008, which is in its entirety herein incorporated by reference.

### FIELD OF INVENTION

A novel nut or seed butter product is described which incorporates curcumin and/or curcuminoids and/or turmeric components and/or turmeric//mixtures hereinafter (cu/co/tc/tu/mx). This product allows for ready incorporation of one or more of these curcumin and/or curcuminoids and/or turmeric components cu/co/tc/tu/mx into the western diet using combinations of nut/seed butters and cu/co/tc/tu/mx is described in this invention. In this invention there are several synergistic advantages: (a) The nut butter medium improves the solubility of cu/co/tc/tu/mx (b) The addition of cu/co/tc/tu/mx to the nut butter produces a product with vastly improved health benefits based on previous medical studies of the curcumin and/or curcuminoids and/or turmeric components. (c) The addition of cu/co/tc/tu/mx to the nut butter significantly reduces oil separation, improves the texture and spreadability of the nut butter (d) The addition of cu/co/tc/tu/mx and natural additives produces a spread and dip that is much tastier than the parent nut butter or cu/co/tc/tu/mx (e) cu/co/tc/tu/mx are powerful antioxidants, they serve to improve the oxidative stability of the nut butters (f) cu/co/tc/tu/mx have antibacterial properties, they protect the nut butter from potential bacterial contamination.

### BACKGROUND OF THE INVENTION

Turmeric is the dried powdered rhizome derived from the plant Curcuma Longa. Curcuminoids constitute around 5% of most turmeric preparations and can be readily isolated from the plant [1]. There has recently been considerable interest in curcumin, [(1E,6E)-1,7-bis (4-hydroxy-3-methoxyphenyl) hepta-1,6-diene 3,5-dione] the primary active ingredient in turmeric, because it has been shown to have anticancer[1,2,3a,3b], antioxidant [4,5], anti-inflammatory [6], anti-Alzheimer's disease activity [7,8] antibacterial activity [9] and cardioprotective benefits [10,11]. Curcumin's anti-cancer properties are a result of it's ability to modulate and work on multiple cell signaling pathways, including cell cycle (cyclin D1 and cyclin E), apoptosis (activation of caspases and down-regulation of antiapoptotic gene products), reduces proliferation (HER-2, EGFR, and AP-1), survival (PI3K/AKT pathway), reduces invasion (MMP-9 and adhesion mols.), angiogenesis (VEGF), metastasis (CXCR-4) and reduces inflammation (NF-kβ, TNF, IL-6, IL-1, COX-2, and 5-LOX) [1,2,3,4]. Curcumin has multiple neuro-protective mechanisms including inhibition of inflammation, suppression of (Aymyloid) Aβ production, reduction of reactive oxygen species by chelating metals, inhibition of stress pathways and induction of heat shock proteins [7]. There is evidence that that turmeric consumption in the diet (South East Asian) has been correlated with a reduced incidence of Alzheimer's disease [7] and colon cancer [12]. It is thus evident from the above that the making curcumin and/or curcuminoids and/or turmeric components intake an integral part of the American diet will have a huge positive impact on American health and healthcare. Turmeric, Curcumin and Curcuminoids are Generally Regarded as Safe for human consumption by the Unites States Food and Drug Administration. There have been over 2600 peer reviewed articles published in English since 1966, ˜19 review articles have been written in the last ten years and curcumin has been or is currently being used in over 17 clinical trials [4].

U.S. Pat. No. 5,891,924 invented by Aggarwal, B. B entitled “Curcumin (diferuloylmethane) inhibition of NF.kappa.B activation” describes a method of inhibiting the activation of NF kappa B transcription factor using curcumin.

U.S. Pat. No. 6,653,327 invented by Majeed et al. entitled “Cross-regulin composition of turmeric-derived tetrahydrocurcuminoids for skin lightening and protection against UVB rays” describes a cross-regulin composition of turmeric derived tetrahydrocurcumin (THC) for skin lightening and protection against UVB rays. Invention disclosures on the use of curcumin in disease treatment include IND numbers 51,685; 67,963; 65,700/S-000; and 71.

A representative example of previous invention disclosures of bread spreads is U.S. Pat. No. 5,366,754 invented by Rudan et al. entitled “Reduced fat peanut butter product and method of making” This involves the composition and method of making a reduced fat high moisture peanut butter. This composition is very different from that of this instant invention because the above invention does not contain any cu/co/tc/tu/mx.

U.S. Pat. No. 4,004,037, invented by Connick, F. G entitled “Peanut butter manufacture” refers to a peanut butter with improved flavor and reduced oxidation and consequently a longer shelf life. Here, the author grinds the peanuts in presence of solid carbon dioxide, so there is less oxygen in the nut butter. They reduce the oxidation of the peanut butter (not other nut butters) by a physical method of replacing oxygen by carbon dioxide, not by adding an antioxidant. This is very different from this instant invention where the oxidation is reduced chemically by curcumin/curcuminoids/turmeric/mixture of these.

WO 2009056252, invented by Mulder, E et al. entitled “Soup or Sauce Composition” refers to a soup or sauce composition as a vehicle for the incorporation of conjugated linoleic acid (CLA) into the diet. The invention is quite different from the current instant invention; no nuts or nut butters or cu/co/tc/tu/mx is used.

U.S. Pat. No. 7,344,747, invented by Perlman, D entitled “Oxidative stabilization of omega-3 fatty acids in low linoleic acid-containing peanut butter” describes a food composition in which omega-3 fatty acids are stabilized against oxidation. Here, the peanut butter is used as a medium to reduce the oxidation of the omega-3 fatty acid which is prone to oxidation by itself. This is very different from the instant invention where cu/co/tc/tu/mx protects a nut butter from oxidation and produces a nut butter with enhanced cu/tc/tu/mx solubility, resistance to oxidation, resistance to bacterial contamination, improved spreadability and vastly enhanced taste and health benefits.

U.S. Pat. No. 4,344,969, invented by Youngquist, R. W et al. entitled “Single-dough cookies having storage stable texture” describes single dough cookies with improved shelf life. This is done by the addition of a carbohydrase enzyme to the cookie.

Currently, there are two ways of incorporating cu/co/tc/tu/mx into the American diet (a) Turmeric extract and curcumin food supplements are available in capsule form in order to provide nutritional and health benefits of curcumin. This capsule method of curcumin/turmeric/curcuminoid intake has the shortcoming that the person has to remember to take them. Most people do not like the idea of taking tablets and after a few days stop eating the supplement altogether. Another shortcoming of Curcumin and turmeric extract in a tablet form is the poor water solubility of cu/co/tu/mx. This is one of reasons for its poor bio-absorption in tablet form (even doses as high as 8 g of curcumin tablets per day administered to human subjects results in an average peak serum concentration of 1.77 microM of curcumin [13]). (b) The second method of incorporating turmeric is consumption of curries. Turmeric is an integral part of the standard daily Indian diet; it is however not used in the daily American or European diet or as part of the diet in many other countries. This method also has the drawback that most turmeric based culinary formulations such as Garam Masala/curry powder/curries are too spicy due to the presence of significant amounts of spices such as red chilli powder and cayenne powder that irritate the western palate which is more used to milder flavors. Hence there is a need for a method of incorporation of cu/co/tc/tu/mx into the diet of Americans in a tasty medium that solubilizes cu/co/tu/mx. The basic idea is that a healthy food can only have a positive impact on human health only if it is very tasty; otherwise only a small fraction of humanity would eat it and not on a consistent basis.

### SUMMARY OF INVENTION

A new medium/carrier for incorporating cu/co/tc/tu/mx into the American diet using combinations of nut/seed butters and cu/co/tc/tu/mx is described in this invention. There is a tremendous need for incorporating cu/co/tc/tu/mx into the American diet because of the health benefits of cu/co/tu/mx, they modulate several biochemical pathways that are beneficial for cancer prevention, prevention of Alzhiemer's disease and reduction in inflammatory diseases. Turmeric is the dried powdered rhizome of the plant Curcuma Longa. Curcuminoids constitute around 5% of most turmeric preparations and can be readily isolated from the plant [1,3a,4]. There has recently been considerable interest in curcumin, [(1E,6E)-1,7-bis(4-hydroxy-3-methoxyphenyl)hepta-1,6-diene 3,5-dione] the primary active ingredient in turmeric, because it has been shown to have anticancer[1,2,3], antioxidant [4,5], anti-inflammatory [6], anti-Alzheimer's disease activity [7,8] and antibacterial activity [9]. There is epidemiological evidence that that turmeric consumption in the diet (South East Asian) correlates with a reduced incidence of Alzheimer's disease [7] and colon cancer [12]. It is thus evident that the current invention which makes cu/co/tc/tu/mx intake an integral part of the American diet will have a huge positive impact on American nutrition, health and healthcare.

Turmeric extract and curcumin food supplements are available in capsule form in order to provide nutritional and health benefits of curcumin. This capsule method of curcumin/turmeric/curcuminoid intake has the shortcoming that the person has to remember to take them. Most people do not like the idea of taking tablets and after a few days stop eating the supplement altogether. Another shortcoming of Curcumin and turmeric extract in a tablet form is the poor water solublilty of cu/co/tu/mx. This is one of reasons for its poor bio-absorption in tablet form (even doses as high as 8 g of curcumin tablets per day administered to human subjects results in an average peak serum concentration of 1.77 microM of curcumin [13]).

Alternatively, nutritional supplements can be incorporated into food products. In the current invention we have used nut butters as a medium for improving the solubility of cu/co/tc/tu/mx which hydrophobic and are much more soluble in the lipid rich nut butter medium thereby potentially improving its bioavailability. The addition of cu/co/tc/tu/mx to the nut butters and in some embodiments other spices and sweeteners such has honey results in a product which is vastly tastier than both the starting nut butter and cu/co/tc/tu/mx. In these unique formulations there are several synergistic advantages creating a novel, very useful product: (a) The addition of cu/co/tc/tu/mx produces a novel product with vastly improved health benefits compared to the parent nut butter (b) The nut butter medium improves the solubility and hence potentially the bioabsorption of cu/co/tc/tu/mx (c) The addition of cu/co/tc/tu/mx and spices and sweetener to the nut butter significantly reduces oil separation. Oil separation is a problem with all natural nut butters, the current product has highly reduced oil separation and improved texture and spreadability compared to the starting nut butter (d) The addition of cu/co/tc/tu/mx to the nut butters in some embodiments spices produces a spread and dip that is much tastier than both the parent nut butter and cu/co/tc/tu/mx (e) cu/co/tc/tu/mx are excellent antioxidants, they serve to improve the oxidative stability of the nut butters such as almond butter that has a large amount of unsaturated fats that are susceptible to oxidation (f) cu/co/tc/tu/mx have antibacterial properties [9], thereby protecting the nut butter from potential bacterial contamination from some common strains of bacteria.

Here, we have used nut and seed butters as a medium to incorporate cu/co/tc/tu/mx into the American diet while improving the flavor and texture of nut butters and improving the shelf life. (Nuts/seeds/powdered nuts//nut butters/seed butters/powdered seeds/mixtures of these are represented as nu/se/pn/nb/sb/ps/mns) in this document.). cu/co/tu/mx is extremely beneficial for the health and it is essential that it be made part of the daily diet of Americans. This would potentially mean reduced colon cancer rates, and lowered rates of many other diseases such as Alzheimer's disease and other inflammatory diseases. One method of administering turmeric is eating it as curry (curry does not have pure curcumin or curcuminoids, it contains an ill-defined/varying amount of turmeric with several other spices). This approach has shortcomings: Most Indians consume turmeric as curry but curry powder contains large amounts of hot spices such as red chillies which is irritate the American palate. Hence, advocating Americans, (most of whom are used to milder flavors) to eat curries is not an effective way of incorporating turmeric to the American diet at large. Most Americans are used to nut butters and it is an integral part of the American diet just as turmeric is part of the Indian diet. (Nut butters are not part of the Indian Diet). One can make a significant positive impact on human health only if a health food is very tasty; otherwise only a small fraction of humanity would eat it and not on a consistent basis; In the preferred embodiments of the current invention, the scientifically designed cu/co/tc/tu/mx spread with nut butters provides a well defined quantity of cu/co/tc/tu/mx per serving (500-800 mg). It is very delicious and taste perfected for the western palate.

Formulations and manufacturing procedures for (P) wherein P is a novel cu/co/tc/tu/mx and nut/seed butter based spread and dip with or without other additives. P has enhanced health benefits, shelf life and taste compared to the starting nut butter. P serves as a novel, improved and general method for incorporating cu/co/tc/tu/mx into the daily American diet. P is defined by equation (i).

nu/se/pn/nb/sb/ps/mns+cu/co/tc/tu/mx+V+W+X+Y+Z→P (i)

Z=Z′+Z″+Z′″

wherein nuts, powdered nuts and nut butters in nu/se/pn/nb/sb/ps/mns include but are not limited to almonds, peanuts, pistachios, cashews, macademia, pine, walnut, hazelnut, chestnut, brazilnut, soynut, filberts, hickory, pecans in ground, powdered, crunchy, smooth, paste, whole, crushed, broken or slurry form. The seeds and seed butters include but are not limited to the following: sesame seeds, sunflower seeds or other seeds in ground, powdered, crunchy, smooth, paste, whole, crushed, broken or slurry form. Either one of the nuts or seeds or a combination of two or more types of nuts or seeds or mixtures of nuts and seeds may be used.

V=none, one or more of spices including but not limited to garlic, black pepper, white pepper, pepper corns, clove, cumin, nutmeg, mace, thyme, cinnamon, coriander, oregano, parsley, basil, holy basil, allspice, cilantro, rosemary, aniseed, fennel, dill (seed, herb or weed), cardamom, paprika, saffron, marjoram, mint, fenugreek, ginger, star anise, pink pepper, long pepper, sage, or other spice, powdered, whole or as a paste or mixtures thereof. In addition V includes spices selected from the following or mixtures thereof, these may be used in less preferred embodiments: Ajwain, Akudjura, alexanders, alkanet, allspice, galangal, amchur (mango powder), anise, aniseed myrtle, annato, apple mint, azafoetida, avocado leaf, barberry, bay leaf, borage, black cardamom, black mustard, blue fenugreek, brown mustard, calamint, calendula, candlenut, capers, caraway, catnip, cassia, cayenne pepper, celery seed, chamomile, chervil, chicory, chili pepper, chilli powder, chipotle, chives, cicely, cilantro, cinnamon myrtle, clary, clary sage, cost mary, cuban oregano, cubeb pepper, cud weed, culantro, curry leaf, curry plant, dorrigo pepper, elder flower, epazote, culantro (long coriander), fingerroot, french sorel, galingale, garlic chives, ginkgo nuts, golpar, grains of paradise, grains of selim, green tea, ground ivy, hops, horseradish, houttuynia, hot pepper, hyssop, jasmine, juniper berry, jalepenos, kaffir lime, kaffir lime leaves, lokam, lavender, laser (silphium), lemon balm, lemon basil, lemon grass, lemon mint, lemon myrtle, lemon thyme, lemon verbena, lesser galangal, licorice, lime flower, linden flower, rice paddy herb, long pepper, lovage, luohanguo, mace, marjoram, nasturtium, nigella, black caraway, olida, orris root, sweet cicely, pandan flower (kewra), pandan leaf, paprika, paracress, peppermint, peppermint gum, perilla, piper sarmentosum, white pumpkin, poppy seed, ramsons (wood garlic), red pepper, ribbery, rue, safflower, saigon cinnamon, salad Burnet, salep, sassafras, savory, sesame seed, sheep's sorrel, sichuan pepper, sloe berries, sorrel, spearmint, spikenard, sumac, summer savory, sweet woodruff, talinum, tamarind, tansy, tarragon, tasmanian pepper, tea, felty germander, that basil, tulsi, vanilla, vietnameese balm, vietnamese cinnamon, vietnamese corriander, wasabi, water-pepper, water cress, wattle seed, white mustard, wild thyme, winter savory, wintergreen, wood avens, woodruff, wormwood, yellow mustard, yerba buena, zaatar herb, zedoary, lemon, celery, celery seed, nigella seed, poppy seed, pimenta, pimento, orange peel, rapeseed, nori, mustard seeds, pimento, scallions, sweet pepper. Spice mixtures including but not limited to berbere, curry powder, chinese five spice powder, garam masala, chaat masala, goda masala, Herbes de Provence, kaala masala, Khmeli suneli, pudding spice, panch phoron, pumpkin pie spice, quatre epices, rasel hanout, tandoori masala, zaatar, jerk spice.

W=none, one or more of sweetening agents including but not limited to honey, sugar (including but not limited to cane sugar, beet sugar, barley sugar), brown sugar, raw sugar (including but not limited to turbinado sugar, jaggery, muscovedo, panela, sucanat) palm sugar, amazake, caramel, molasses, maple syrup, taffy, agave nectar, corn syrup, birch syrup, pine syrup, hickory syrup, poplar syrup, palm syrup, sugar beet syrup, sorghum syrup, cane syrup, cane sugar, golden syrup, barley malt syrup, rice syrup, agave syrup, yacon syrup, fruit powder or paste, fruit juice, fruit and vegetable sugars (including but not limited to pumpkin sugar, watermelon sugar, date sugar, pekmez) vegetables or vegetable extract including but not limited to carrots, beets, celery in ground, paste, juice, grated or other form, sugar cane juice, carbohydrates including but not limited to glucose, dextrose, fructose, levulose, invert sugar/invert syrup, galactose, sucrose, lactose or mixtures thereof, stevia, Jiaogulan, sugar substitutes (including but not limited to acesulfame potassium, salt of aspartame-acesulfame, acesulfame, alitame, aspartame (equal or nutraseweet), anethole, cyclamate, glycyrrhizin, lo han guo, neotame, perillartine, saccharin, stevioside, sucralose, inulin, or mixtures thereof), sugar alcohols (including but not limited to glycol, glycerol, erythritol, threitol, arabitol, xylitol, ribitol, mannitol, sorbitol, dulcitol, iditol, isomalt, maltitol, lactitol, polyglycitol or mixtures thereof), natural sweeteners (including but not limited to brazzein, curculin, hydrogenated starch hydrolysates, mabinlin, miraculin, monatin, monellin, pentadin, tagatose, thaumatin or mixtures thereof) and mixtures thereof.

X=none, one or more of salt or salt substitutes such as salt (NaCl), sea salt, rock salt, potassium Chloride, potassium lactate, black salt, pink salt, smoked salt or other salt or salt substitute. The preferred embodiments employ sodium chloride, sea salt and/or rock salt.

Y=none, one or more of oils including but not limited to Peanut oil, walnut oil, almond oil, cotton seed oil, olive oil, corn oil, canola oil, ghee, butter, margarine, coconut oil, saffola oil, sunflower oil, mustard oil, cashew oil, palm oil, soybean oil, rapeseed oil, hazelnut oil, pecan oil, pine nut oil, other nut oils, linseed oils, rice bran oil, fish oil, margarine, shortening, partially or fully hydrogenated oils or fats, mono and diglycerides, hazelnut oil, macademia nut oil, mongongo nut oil (manketti oil), pine nut oil, pistachio oil, pumpkin seed, watermelon, sesame or other seed oils, apricot oil, amaranth oil, apple seed oil, argan oil, artichoke oil, avocado oil, babassu oil, ben oil borneo tallow nut oil, cape chestnut oil, cocoa butter, carob pod oil, coriander seed oil, dika oil, flax oil, false flax seed oil, grape seed oil, kapok seed oil, pine nut oil, lard, lallemantia oil, jojoba oil, manila oil, meadofoam seed oil, nutmeg butter, okra seed oil, papaya seed oil, preilla seed oil, pequi oil, poppy seed oil, primrose oil, prune kernel oil, quinoa oil, ramtil oil, royle oil, sacha-inchi oil, soy oil, tea seed oil, thisle oil, tomato seed oil wheat germ oil, mono or di glycerides, or other oils, fats or oil substitutes or mixtures thereof.

Z′=none, one or more of acidulents or acidity modifiers including but not limited to citric acid, vinegar, lemon juice, dilute acetic acid, tartaric acid, lactic acid, malic acid, lime juice, acidity modifiers including but not limited to sodium bicarbonate, sodium carbonate or the salts of acids including acetic, tartaric, lactic, malic, citric acids or mixtures thereof.

Z″=none, one or more of grains, pulses, tubers or starches including but not limited to wheat, wheat germ, broken wheat, rice, corn, potato, barley, maize, sorghum, oats, millets, rye, triticale, buckwheat, bread crumbs, corn crumbs, corn starch, katakuri starch, sago, tapioca, potato, arrow root or other tubers, other starches, fonio, quinoa, spelt, amaranth, kaniwa, durum, semolina, pulses including but not limited to kidney beans, black beans, mung bean, black gram, dal, garbanzo beans, lima bean, fava bean, lentils, peas including but not limited to green pea, yellow pea, cow pea, or other grains, tubers, peas or beans in whole, powdered or ground form or as a paste or mixtures thereof.

Z″′=none, one or more of the following: fruit or fruit paste or vegetables including but not limited to raisins, prunes, apples, apricots, berries, amla (Indian gooseberry), jamoon, dates, citron, figs, cranberries or other fruit or vegetables including but not limited to carrots, or other vegetables in ground, juice, grated or other form, eggs, egg yolks, egg whites or egg substitutes, cheese, whey, milk solids, yoghurt or other milk substitute, vitamins, amino acids, proteins, minerals or herbs (culinary or medicinal, including but not limited to chamomile, psyllium, brahmi, pippali [long pepper], bael, tea (including but not limited to green, black) aloe, satavari, asparagus, eucalyptus, sandalwood, saw palmetto, neem) plants or extracts of herbs or plants (including but not limited to reserveratrol, green tea extract, bromelain, pomegranate extract) vegemite, wheat gluten, gluten, glycerol mono stearate, yeast, yeast extract, camphor, food additives (including but not limited to silicon dioxide, silicates, stearic acid, tartaric acid; emulsifiers including but not limited to lecithin, polysorbate 20, flavor enhancers including but not limited to MSG, glutamic acid, inosinic acid, maltitol; humectants including but not limited to urea, quillaaia, desiccants, stabilizers including but not limited to agar, pectin, texture additives including but not limited to collagen, gelatin carrageenan, alginin, guar Dim, locust bean gum, xanthan gum, flavors, chocolate, cocoa, preservatives including but not limited to sodium benzoate, benzoic acid, calcium propionate, potassium sorbate, nitrates, nitrites, sulfites, bisulfites, disodium EDTA, BHA, BHT or colors including but not limited to FD&C blue no 1, 2, green 3, red 3, 40, yellow 5, 6. The amount components Z (Z′+Z″+Z′″) may range from 0-40%.

P=the product obtained, the spread containing nut/seed butters and cu/co/tc/tu/mx. *The manufacturing process include approaches in which (a) the nuts, curcumin and/or curcuminoids and/or turmeric and/or turmeric components and other ingredients (if any) are ground in a grinding, milling, chopping or nut butter producing machine or any another machine that breaks down the ingredients. It should be noted that the other ingredients could be none, one or more of V, W, X, Y, and/or Z in the equation (b) one or more of the nuts/seeds is premade into a nut/seed butter and mixed with cu/co/tc/tu/mx and other ingredients (if any) and ground/blended. The other ingredients could be none, one or more of V, W, X, Y and/or Z. Either of the processes may involve optional heating of one or more of the ingredients.

### BRIEF DESCRIPTION OF THE FIGURES

FIG. 1 is a plot of the Antioxidant Values of a control (almond butter) and sample P3 [Oxygen Radical Absorbing Capacity, (ORAC, [μM Trolox)]

FIG. 2 is a photograph of the filtrates obtained by dissolving curcumin (1 g/100 ml) in almond oil and water. The almond oil has a much higher concentration of dissolved curcumin and water has a negligible concentration of dissolved curcumin. (A) Almond oil (B) water.

### DETAILED DESCRIPTION OF THE INVENTION

A large body of scientific literature supports the potential disease preventing properties of turmeric (curcuma longa) and its active ingredients (curcuminoids, Curcumin is the most important component). South east Asian populations include turmeric regularly in the form of curries and have reduced rates of colon cancer [12] and many other diseases. However, eating curries is not an option for most Americans since curry powder and curries often contain large amount of spices such as red chillies or cayenne pepper. The hot spices irritate the American palate and it is not practical to suggest consumption of curry as a viable method of incorporating Curcumin into the American Diet.

The other alternative is to take pills containing Curcumin or turmeric. This method has the shortcoming that most people do not enjoy taking pills and after a few days or months stop doing so altogether. Thus there is a need for a product which can incorporate Curcumin/turmeric into the diets of Americans in a consistent fashion. This is possible only if the incorporation vehicle is a tasty food item.

A novel product for incorporating curcumin and/or curcuminoids and/or turmeric components/mixtures containing one or more of these cu/co/tc/tu/mx into the American diet using combinations of nut/seed butters and cu/co/tc/tu/mx is described in this invention. In this invention there are several synergistic advantages: (a) The nut butter medium improves the solubility of cu/co/tc/tu/mx (b) The addition of cu/co/tc/tu/mx to the nut butter produces a product with vastly improved health benefits. (c) The addition of cu/co/tc/tu/mx to the nut butter significantly reduces oil separation, improves the texture and spreadability of the nut butter (d) The addition of cu/co/tc/tu/mx and natural additives produces a spread and dip that is much tastier than the parent nut butter or cu/co/tc/tu/mx (e) cu/co/tc/tu/mx are powerful antioxidants, they serve to improve the oxidative stability of the nut butters (f) cu/co/tc/tu/mx have antibacterial properties, they protect the nut butter from potential bacterial contamination.

In the current invention we have used nut butters as a medium for improving the solubility of curcumin/curminoids/turmeric components/turmeric (cu/co/tu/mx) which are hydrophobic (not water soluble) and are much more soluble in the lipid rich nut butter medium thereby improving its bioavailability. The addition of cu/co/tc/tu/mx to the nut butters and in some embodiments other spices and sweeteners such has honey results in a product which is vastly tastier than both the starting nut butter and Turmeric/curcumin. In these unique formulations there are several synergistic advantages creating a novel, very useful product: (a) The nut butter medium improves the solubility and hence potentially the bioabsorption of cu/co/tc/tu/mx (b) The addition of cu/co/tc/tu/mx produces a novel product with vastly improved health benefits compared to the parent nut butter. (c) The addition of cu/co/tc/tu/mx and spices and sweetener to the nut butter significantly reduces oil separation, oil separation is a problem with all natural nut butters, the current product has highly reduced oil separation and improved texture and spreadability compared to the starting nut butter (d) The addition of cu/co/tc/tu/mx and spices produces a spread and dip that is much tastier than both the parent nut butter and cu/co/tc/tu/mx by themselves (curcumin/turmeric has a very strong and disagreeable flavor when eaten without other ingredients) (e) cu/co/tc/tu/mx are excellent antioxidants, they serve to improve the oxidative stability of the nut butters such as almond butter that has a large amount of unsaturated fats that are susceptible to oxidation (f) cu/co/tc/tu/mx have antibacterial properties [9], thereby protecting the nut butter from potential bacterial contamination from some common strains of bacteria.

Here, we have used nut and seed butters as a medium to incorporate cu/co/tc/tu/mx into the American diet while improving the flavor and texture of nut butters and improving the shelf life of the nut/seed butter. Turmeric is an integral part of the standard daily Indian diet, it is however not used in the daily American or European diet or as part of the diet in many other countries. The major reasons for the lack of turmeric/curcumin use in the western diet are (a) the fact that most turmeric based culinary formulations such as Garam Masala/curry powder/curries are too spicy due to the presence of significant amounts of spices such as red chilli powder and cayenne powder that irritate the western palate which is more used to milder flavors. (b) the lack of knowledge of its health benefits. It is the primary objective of this invention to produce a bread spread/dip incorporating /turmeric/curcumin/curcuminoids and ground nuts/nut butters which is formulated to appeal to the western palate which imparts the health benefits of regular turmeric/curcumin consumption. It is another objective of this invention to blend turmeric/curcumin in the nut butter medium that will improve its flavor, its solubility and potentially its bioabsorption. It should be noted that turmeric/curcumin/curcuminoids are not water soluble whereas they have improved solubility in a nut butter medium. The basic philosophy behind this invention is that one can make a healthy food can only have a positive impact on human health if it is very tasty; otherwise only a small fraction of humanity would eat it and not on a consistent basis.

This invention involves formulations and manufacturing procedures for (P) where P is a novel cu/co/tc/tu/mx and nut/seed butter based spread and dip with or without other additives. P has enhanced health benefits, shelf life and taste compared to the starting nut butter. P serves as a novel, improved and general method for incorporating cu/co/tc/tu/mx into the daily American diet. P is defined by equation (i).

nu/se/pn/nb/sb/ps/mns+cu/co/tc/tu/mx+V+W+X+Y+Z→P (i)

Z=Z′+Z″+Z′″

wherein nuts, powdered nuts and nut butters in nu/se/pn/nb/sb/ps/mns include but are not limited to almonds, peanuts, pistachios, cashews, macademia, pine, walnut, hazelnut, chestnut, brazilnut, soynut, filberts, hickory, pecans in ground, powdered, butter, crunchy, smooth, paste, whole, crushed, broken or slurry form. The seeds, powdered seeds and seed butters can in include the following but are not limited to the following: sesame seeds, sunflower seeds or other seeds in ground, powdered, crunchy, smooth, paste, whole, crushed, broken or slurry form. Either one of the nuts or seeds or a combination of two or more types of nuts or seeds or mixtures of nuts and seeds may be used.

wherein V=none, one or more of spices including but not limited to garlic, black pepper, white pepper, pepper corns, clove, cumin, nutmeg, mace, thyme, cinnamon, coriander, oregano, parsley, basil, holy basil, allspice, cilantro, rosemary, aniseed, fennel, dill (seed, herb or weed), cardamom, paprika, saffron, marjoram, mint, fenugreek, ginger, star anise, pink pepper, long pepper, sage, or other spice, powdered, whole or as a paste or mixtures thereof. In addition V includes spices selected from the following or mixtures thereof, these may be used in less preferred embodiments: Ajwain, Akudjura, alexanders, alkanet, allspice, galangal, amchur (mango powder), anise, aniseed myrtle, annato, apple mint, azafoetida, avocado leaf, barberry, bay leaf, borage, black cardamom, black mustard, blue fenugreek, brown mustard, calamint, calendula, candlenut, capers, caraway, catnip, cassia, cayenne pepper, celery seed, chamomile, chervil, chicory, chili pepper, chilli powder, chipotle, chives, cicely, cilantro, cinnamon myrtle, clary, clary sage, cost mary, cuban oregano, cubeb pepper, cud weed, culantro, curry leaf, curry plant, dorrigo pepper, elder flower, epazote, culantro (long coriander), fingerroot, french sorel, galingale, garlic chives, ginkgo nuts, golpar, grains of paradise, grains of selim, green tea, ground ivy, hops, horseradish, houttuynia, hot pepper, hyssop, jasmine, juniper berry, jalepenos, kaffir lime, kaffir lime leaves, lokam, lavender, laser (silphium), lemon balm, lemon basil, lemon grass, lemon mint, lemon myrtle, lemon thyme, lemon verbena, lesser galangal, licorice, lime flower, linden flower, rice paddy herb, long pepper, lovage, luohanguo, mace, marjoram, nasturtium, nigella, black caraway, olida, orris root, sweet cicely, pandan flower (kewra), pandan leaf, paprika, paracress, peppermint, peppermint gum, perilla, piper sarmentosum, white pumpkin, poppy seed, ramsons (wood garlic), red pepper, ribbery, rue, safflower, saigon cinnamon, salad burnet, salep, sassafras, savory, sesame seed, sheep's sorrel, sichuan pepper, sloe berries, sorrel, spearmint, spikenard, sumac, summer savory, sweet woodruff, talinum, tamarind, tansy, tarragon, tasmanian pepper, tea, felty germander, that basil, tulsi, vanilla, vietnameese balm, vietnamese cinnamon, vietnamese corriander, wasabi, water-pepper, water cress, wattle seed, white mustard, wild thyme, winter savory, wintergreen, wood avens, woodruff, wormwood, yellow mustard, yerba buena, zaatar herb, zedoary, lemon, celery, celery seed, nigella seed, poppy seed, pimenta, pimento, orange peel, rapeseed, nori, mustard seeds, pimento, scallions, sweet pepper. Spice mixtures including but not limited to berbere, curry powder, chinese five spice powder, garam masala, chaat masala, goda masala, Herbes de Provence, kaala masala, Khmeli suneli, pudding spice, panch phoron, pumpkin pie spice, quatre epices, rasel hanout, tandoori masala, zaatar, jerk spice.

Among the spices the embodiment including black./white pepper and/or garlic is the most preferred spice combination in the embodiment.

wherein W=none, one or more of sweetening agents including but not limited to honey, sugar (including but not limited to cane sugar, beet sugar, barley sugar), brown sugar, raw sugar (including but not limited to turbinado sugar, jaggery, muscovedo, panela, sucanat) palm sugar, amazake, caramel, molasses, maple syrup, taffy, agave nectar, corn syrup, birch syrup, pine syrup, hickory syrup, poplar syrup, palm syrup, sugar beet syrup, sorghum syrup, cane syrup, cane sugar, golden syrup, barley malt syrup, rice syrup, agave syrup, yacon syrup, fruit powder or paste, fruit juice, fruit and vegetable sugars (including but not limited to pumpkin sugar, watermelon sugar, date sugar, pekmez) vegetables or vegetable extract including but not limited to carrots, beets, celery in ground, paste, juice, grated or other form, sugar cane juice, carbohydrates including but not limited to glucose, dextrose, fructose, levulose, invert sugar/invert syrup, galactose, sucrose, lactose or mixtures thereof, stevia, Jiaogulan, sugar substitutes (including but not limited to acesulfame potassium, salt of aspartame-acesulfame, acesulfame, alitame, aspartame (equal or nutraseweet), anethole, cyclamate, glycyrrhizin, lo han guo, neotame, perillartine, saccharin, stevioside, sucralose, inulin, or mixtures thereof), sugar alcohols (including but not limited to glycol, glycerol, erythritol, threitol, arabitol, xylitol, ribitol, mannitol, sorbitol, dulcitol, iditol, isomalt, maltitol, lactitol, polyglycitol or mixtures thereof), natural sweeteners (including but not limited to brazzein, curculin, hydrogenated starch hydrolysates, mabinlin, miraculin, monatin, monellin, pentadin, tagatose, thaumatin or mixtures thereof) and mixtures thereof. The preferred embodiments include those employing honey, molasses, or agave nectar.

wherein X=none, one or more of salt or salt substitutes such as salt (NaCl), sea salt, rock salt, potassium Chloride, potassium lactate, black salt, pink salt, smoked salt or other salt or salt substitute. The preferred embodiments employ sodium chloride, sea salt and/or rock salt.

wherein Y=none, one or more of oils including but not limited to Peanut oil, walnut oil, almond oil, cotton seed oil, olive oil, corn oil, canola oil, ghee, butter, margarine, coconut oil, saffola oil, sunflower oil, mustard oil, cashew oil, palm oil, soybean oil, rapeseed oil, hazelnut oil, pecan oil, pine nut oil, other nut oils, linseed oils, rice bran oil, fish oil, margarine, shortening, partially or fully hydrogenated oils or fats, mono and diglycerides, hazelnut oil, macademia nut oil, mongongo nut oil (manketti oil), pine nut oil, pistachio oil, pumpkin seed, watermelon, sesame or other seed oils, apricot oil, amaranth oil, apple seed oil, argan oil, artichoke oil, avocado oil, babassu oil, ben oil borneo tallow nut oil, cape chestnut oil, cocoa butter, carob pod oil, coriander seed oil, dika oil, flax oil, false flax seed oil, grape seed oil, kapok seed oil, pine nut oil, lard, lallemantia oil, jojoba oil, manila oil, meadofoam seed oil, nutmeg butter, okra seed oil, papaya seed oil, preilla seed oil, pequi oil, poppy seed oil, primrose oil, prune kernel oil, quinoa oil, ramtil oil, royle oil, sacha-inchi oil, soy oil, tea seed oil, thisle oil, tomato seed oil wheat germ oil, mono or di glycerides, or other oils, fats or oil substitutes or mixtures thereof. The preferred composition employs almond oil, peanut oil, olive oil or walnut oil.

wherein Z′=none, one or more of acidulents or acidity modifiers including but not limited to citric acid, vinegar, lemon juice, dilute acetic acid, tartaric acid, lactic acid, malic acid, lime juice, acidity modifiers including but not limited to sodium bicarbonate, sodium carbonate or the salts of acids including acetic, tartaric, lactic, malic, citric acids or mixtures thereof.

Z″ none, one or more of grains, pulses, tubers or starches including but not limited to wheat, wheat germ, broken wheat, rice, corn, potato, barley, maize, sorghum, oats, millets, rye, triticale, buckwheat, bread crumbs, corn crumbs, corn starch, katakuri starch, sago, tapioca, potato, arrow root or other tubers, other starches, fonio, quinoa, spelt, amaranth, kaniwa, durum, semolina, pulses including but not limited to kidney beans, black beans, mung bean, black gram, dal, garbanzo beans, lima bean, fava bean, lentils, peas including but not limited to green pea, yellow pea, cow pea, or other grains, tubers, peas or beans in whole, powdered or ground form or as a paste or mixtures thereof.

Z′″=none, one or more of the following: fruit or fruit paste or vegetables including but not limited to raisins, prunes, apples, apricots, berries, amla (Indian gooseberry), jamoon, dates, citron, figs, cranberries or other fruit or vegetables including but not limited to carrots, or other vegetables in ground, juice, grated or other form, eggs, egg yolks, egg whites or egg substitutes, cheese, whey, milk solids, yoghurt or other milk substitute, vitamins, amino acids, proteins, minerals or herbs (culinary or medicinal, including but not limited to chamomile, psyllium, brahmi, pippali [long pepper], bael, tea (including but not limited to green, black), aloe, satavari, asparagus, eucalyptus, sandalwood, saw palmetto, neem), plants or extracts of herbs or plants (including but not limited to reserveratrol, green tea extract, bromelain, pomegranate extract), vegemite, wheat gluten, gluten, glycerol mono stearate, yeast, yeast extract, camphor, food additives (including but not limited to silicon dioxide, silicates, stearic acid, tartaric acid; emulsifiers including but not limited to lecithin, polysorbate 20, flavor enhancers including but not limited to MSG, glutamic acid, inosinic acid, maltitol; humectants including but not limited to urea, quillaaia, desiccants, stabilizers including but not limited to agar, pectin, texture additives including but not limited to collagen, gelatin carrageenan, alginin, guar gum, locust bean gum, xanthan gum, flavors, chocolate, cocoa, preservatives including but not limited to sodium benzoate, benzoic acid, calcium propionate, potassium sorbate, nitrates, nitrites, sulfites, bisulfites, disodium EDTA, BHA, BHT or colors including but not limited to FD&C blue no 1, 2, green 3, red 3, 40, yellow 5, 6. The amount components Z (Z′+Z″+Z′″) may range from 0-40%.

The preferred embodiment has 0-10% of additives Z (Z′+Z″+Z′″).

Wherein P=the product obtained, the spread containing nut/seed butters and cu/co/tc/tu/mx. The manufacturing process include approaches in which (a) the nuts, curcumin and/or curcuminoids and/or turmeric and/or turmeric components and other ingredients (if any) are ground in a grinding, milling, chopping or nut butter producing machine or any another machine that breaks down the ingredients. It should be noted that the other ingredients could be none, one or more of V, W, X, Y, and/or Z in the equation (b) one or more of the nuts/seeds is premade into a nut/seed butter and mixed with cu/co/tc/tu/mx and other ingredients (if any) and ground/blended. The other ingredients could be none, one or more of V, W, X, Y and/or Z. Either of the processes may involve optional heating of one or more of the ingredients.

The Invention Involves:

1) a composition of raw or roasted nuts or seeds in ground, powdered, crunchy, smooth, paste, whole, crushed, broken or slurry form; the nut/seed component is called component 1 (the nuts including but not limited to almonds, peanuts, pistachios, cashews, macademia, pine, walnut, hazelnut, chestnut, brazilnut, soynut, filberts, hickory, pecans). The seeds include the following but are not limited to: sesame seeds, sunflower seeds or other seeds. Either one of the nuts or seeds or a combination of two or more types of nuts or seeds may be blended/ground with one or more of the following: turmeric, curcumin, curcuminoids or turmeric extract. The curcumin/curcuminoids/turmeric extract or components/turmeric/a mixture of one or more of these cu/co/tc/tu/mx is component 2. Component 1 can range from 30-99.8% by weight. Component 2 can range from 0.2-70% by weight. This composition of components 1 and 2 is called A. Preferred embodiments include almonds, peanuts, cashew nut butters with cu/co/tu/mx. The most preferred embodiment includes almond butter (80-99.8%) and turmeric/curcumin/curcuminoids (0.2-15%).

2) a composition of A with one or more of spices one or more of spices including but not limited to garlic, black pepper, white pepper, pepper corns, clove, cumin, nutmeg, mace, thyme, cinnamon, coriander, oregano, parsley, basil, holy basil, allspice, cilantro, rosemary, aniseed, fennel, dill (seed, herb or weed), cardamom, paprika, saffron, marjoram, mint, fenugreek, ginger, star anise, pink pepper, long pepper, sage, or other spice, powdered, whole or as a paste or mixtures thereof. In addition the spices include spices selected from the following or mixtures thereof, these may be used in less preferred embodiments: Ajwain, Akudjura, alexanders, alkanet, allspice, galangal, amchur (mango powder), anise, aniseed myrtle, annato, apple mint, azafoetida, avocado leaf, barberry, bay leaf, borage, black cardamom, black mustard, blue fenugreek, brown mustard, calamint, calendula, candlenut, capers, caraway, catnip, cassia, cayenne pepper, celery seed, chamomile, chervil, chicory, chili pepper, chilli powder; chipotle, chives, cicely, cilantro, cinnamon myrtle, clary, clary sage, cost mary, cuban oregano, cubeb pepper, cud weed, culantro, curry leaf, curry plant, dorrigo pepper, elder flower, epazote, culantro (long coriander), fingerroot, french sorel, galingale, garlic chives, ginkgo nuts, golpar, grains of paradise, grains of selim, green tea, ground ivy, hops, horseradish, houttuynia, hot pepper, hyssop, jasmine, juniper berry, jalepenos, kaffir lime, kaffir lime leaves, lokam, lavender, laser (silphium), lemon balm, lemon basil, lemon grass, lemon mint, lemon myrtle, lemon thyme, lemon verbena, lesser galangal, licorice, lime flower, linden flower, rice paddy herb, long pepper, lovage, luohanguo, mace, marjoram, nasturtium, nigella, black caraway, olida, orris root, sweet cicely, pandan flower (kewra), pandan leaf, paprika, paracress, peppermint, peppermint gum, perilla, piper sarmentosum, white pumpkin, poppy seed, ramsons (wood garlic), red pepper, ribbery, rue, safflower, saigon cinnamon, salad burnet, salep, sassafras, savory, sesame seed, sheep's sorrel, sichuan pepper, sloe berries, sorrel, spearmint, spikenard, sumac, summer savory, sweet woodruff, talinum, tamarind, tansy, tarragon, tasmanian pepper, tea, felty germander, that basil, tulsi, vanilla, vietnameese balm, vietnamese cinnamon, vietnamese corriander, wasabi, water-pepper, water cress, wattle seed, white mustard, wild thyme, winter savory, wintergreen, wood avens, woodruff, wormwood, yellow mustard, yerba buena, zaatar herb, zedoary, lemon, celery, celery seed, nigella seed, poppy seed, pimenta, pimento, orange peel, rapeseed, nori, mustard seeds, pimento, scallions, sweet pepper. Spice mixtures including but not limited to berbere, curry powder, chinese five spice powder, garam masala, chaat masala, goda masala, Herbes de Provence, kaala masala, Khmeli suneli, pudding spice, panch phoron, pumpkin pie spice, quatre epices, rasel hanout, tandoori masala, zaatar, jerk spice.

A composition of A with one or more spices is referred to as B.

3) A composition of A or B mixed with one or more of sweeteners including but not limited to honey, sugar (including but not limited to cane sugar, beet sugar, barley sugar), brown sugar, raw sugar (including but not limited to turbinado sugar, jaggery, muscovedo, panela, sucanat) palm sugar, amazake, caramel, molasses, maple syrup, taffy, agave nectar, corn syrup, birch syrup, pine syrup, hickory syrup, poplar syrup, palm syrup, sugar beet syrup, sorghum syrup, cane syrup, cane sugar, golden syrup, barley malt syrup, rice syrup, agave syrup, yacon syrup, fruit powder or paste, fruit juice, fruit and vegetable sugars (including but not limited to pumpkin sugar, watermelon sugar, date sugar, pekmez) vegetables or vegetable extract including but not limited to carrots, beets, celery in ground, paste, juice, grated or other form, sugar cane juice, carbohydrates including but not limited to glucose, dextrose, fructose, levulose, invert sugar/invert syrup, galactose, sucrose, lactose or mixtures thereof, stevia, Jiaogulan, sugar substitutes (including but not limited to acesulfame potassium, salt of aspartame-acesulfame, acesulfame, alitame, aspartame (equal or nutraseweet), anethole, cyclamate, glycyrrhizin, lo han guo, neotame, perillartine, saccharin, stevioside, sucralose, inulin, or mixtures thereof), sugar alcohols (including but not limited to glycol, glycerol, erythritol, threitol, arabitol, xylitol, ribitol, mannitol, sorbitol, dulcitol, iditol, isomalt, maltitol, lactitol, polyglycitol or mixtures thereof), natural sweeteners (including but not limited to brazzein, curculin, hydrogenated starch hydrolysates, mabinlin, miraculin, monatin, monellin, pentadin, tagatose, thaumatin or mixtures thereof) and mixtures thereof. This shall be called C.

4) A composition of A or B or C mixed with one or more of the following: salt (NaCl), sea salt, rock salt, potassium Chloride, potassium lactate, black salt, pink salt, smoked salt or other salt or salt substitute. This composition will be referred to as D.

5) A composition of A or B or C or D mixed with one or more oils including but not limited to Peanut oil, walnut oil, almond oil, cotton seed oil, olive oil, corn oil, canola oil, ghee, butter, margarine, coconut oil, saffola oil, sunflower oil, mustard oil, cashew oil, palm oil, soybean oil, rapeseed oil, hazelnut oil, pecan oil, pine nut oil, other nut oils, linseed oils, rice bran oil, fish oil, margarine, shortening, partially or fully hydrogenated oils or fats, mono and diglycerides, hazelnut oil, macademia nut oil, mongongo nut oil (manketti oil), pine nut oil, pistachio oil, pumpkin seed, watermelon, sesame or other seed oils, apricot oil, amaranth oil, apple seed oil, argan oil, artichoke oil, avocado oil, babassu oil, ben oil borneo tallow nut oil, cape chestnut oil, cocoa butter, carob pod oil, coriander seed oil, dika oil, flax oil, false flax seed oil, grape seed oil, kapok seed oil, pine nut oil, lard, lallemantia oil, jojoba oil, marula oil, meadofoam seed oil, nutmeg butter, okra seed oil, papaya seed oil, preilla seed oil, pequi oil, poppy seed oil, primrose oil, prune kernel oil, quinoa oil, ramtil oil, royle oil, sacha-inchi oil, soy oil, tea seed oil, thisle oil, tomato seed oil wheat germ oil, mono or di glycerides, or other oils, fats or oil substitutes or mixtures thereof. This composition is referred to as E.

6) A composition of A or B or C or D or E combined with one or more of additives such as acidulents or acidity modifiers: acidulents include but are not limited to citric acid, vinegar, lemon juice, dilute acetic acid, tartaric acid, lactic acid, malic acid, lime juice, acidity modifiers include but are not limited to sodium bicarbonate, sodium carbonate or the salts of acids including acetic, tartaric, lactic, malic, citric acids or mixtures thereof. This is called F.

7) A composition of A or B or C or D or E or F may be combined with one or more of grains, pulses, tubers or starches including but not limited to wheat, wheat germ, broken wheat, rice, corn, potato, barley, maize, sorghum, oats, millets, rye, triticale, buckwheat, bread crumbs, corn crumbs, corn starch, katakuri starch, sago, tapioca, potato, arrow root or other tubers, other starches, fonio, quinoa, spelt, amaranth, kaniwa, durum, semolina, pulses including but not limited to kidney beans, black beans, mung bean, black gram, dal, garbanzo beans, lima bean, fava bean, lentils, peas including but not limited to green pea, yellow pea, cow pea, or other grains, tubers, peas or beans in whole, powdered or ground form or as a paste or mixtures thereof. This composition is G.

8) A composition of A or B or C or D or E or F or G blended with one or more of the following fruit or fruit paste or vegetables including but not limited to raisins, prunes, apples, apricots, berries, amla (Indian gooseberry), jamoon, dates, citron, figs, cranberries or other fruit or vegetables including but not limited to carrots, or other vegetables in ground, juice, grated or other form, eggs, egg yolks, egg whites or egg substitutes, cheese, whey, milk solids, yoghurt or other milk substitute, vitamins, amino acids, proteins, minerals or herbs (culinary or medicinal, including but not limited to chamomile, psyllium, brahmi, pippali [long pepper], bael, tea (including but not limited to green, black) aloe, satavari, asparagus, eucalyptus, sandalwood, saw palmetto, neem) plants or extracts of herbs or plants (including but not limited to reserveratrol, green tea extract, bromelain, pomegranate extract), vegemite, wheat gluten, gluten, glycerol mono stearate, yeast, yeast extract, camphor, food additives (including but not limited to silicon dioxide, silicates, stearic acid, tartaric acid; emulsifiers including but not limited to lecithin, polysorbate 20, flavor enhancers including but not limited to MSG, glutamic acid, inosinic acid, maltitol; humectants including but not limited to urea, quillaaia, desiccants, stabilizers including but not limited to agar, pectin, texture additives including but not limited to collagen, gelatin carrageenan, alginin, guar gum, locust bean gum, xanthan gum, flavors, chocolate, cocoa, preservatives including but not limited to sodium benzoate, benzoic acid, calcium propionate, potassium sorbate, nitrates, nitrites, sulfites, bisulfites, disodium EDTA, BHA, BHT or colors including but not limited to FD&C blue no 1, 2, green 3, red 3, 40, yellow 5, 6, This is composition H

9) A composition of A or B or C or D or E or F or G or H with improved oxidative stability compared to the parent nut/seed butter (component 1).

10) A composition of A or B or C or D or E or F or G or H with improved resistance to bacterial growth compared to the parent nut/seed butter.

11) A composition of A or B or C or D or E or F or G or H with improved flavor and texture (vastly reduced oil separation; in nut butters such as Almond butter, oil separation is a serious disadvantage) compared to the parent nut/seed butter.

12) A composition of peanut, walnut, pistachio, cashew, almond or mixed nut butter (80-95%), turmeric and/or curcuminoids (1-5%), black and or white pepper (1-5%), Garlic (1-5%), honey (2-10%), peanut/walnut/pistachio/cashew/almond/olive oil or a mixture of oils (0-10%) and salt (0-2%) is a preferred embodiment.

13) A composition of almond butter (75-98%), pistachio, cashew, almond or mixed nut butter (80-95%) curcumin and/or curcuminoids (1-9%), black and or white pepper (1-5%), Garlic (1-5%), honey (2-10%), almond oil (0-10%) and salt (0-2%) is a preferred embodiment.

14) A composition of almond butter (60-98%), turmeric and/or curcuminoids (1-5%), black and or white pepper (1-5%), Garlic (1-5%), honey (2-10%), almond oil (0-10%) and salt (0-2%) is a preferred embodiment.

15) A composition of almond butter (80-95%), turmeric and/or curcuminoids (1-5%), black and or white pepper (1-5%), Garlic (1-5%), honey (2-10%), almond oil (0-10%) and salt (0-2%) is the most preferred embodiment.

16) A composition of walnut butter (60-98%), turmeric and/or curcuminoids (1-5%), black and or white pepper (1-5%), Garlic (1-5%), honey (2-10%), almond oil (0-10%) and salt (0-2%) is a preferred embodiment.

17) A composition of cashew butter (60-98%), turmeric and/or curcuminoids (1-5%), black and or white pepper (1-5%), Garlic (1-5%), honey (2-10%), almond oil (0-10%) and salt (0-2%) is a preferred embodiment.

18) A composition of almond butter (60-98%), curcuminoids and/or curcumin (1-8%), black and or white pepper (1-5%), Garlic (1-5%), honey (2-10%), almond oil (0-10%) and salt (0-2%) is a preferred embodiment.

19) A composition of almond butter (60-98%), curcuminoids and/or curcumin and/or turmeric (1-10%), black and or white pepper (1-5%), Garlic (1-5%), honey (2-10%), almond oil (0-10%) and salt (0-2%) and small amounts (0-9%) of one or more of spices such as cayenne, hot, chilli or other pepper.

20) A composition of almond butter (60-98%), curcuminoids (1-8%), black or white pepper (1-5%), Garlic (1-5%), honey (2-10%), almond oil (0-10%) and salt (0-2%) is a preferred embodiment.

21) A composition of almond butter (80-95%), turmeric and/or curcuminoids (1-5%), black and or white pepper (1-5%), Garlic (1-5%), honey (2-10%), almond oil (0-10%) and salt (0-2%) with improved oxidative stability compared to almond butter.

22) A composition of almond butter (80-95%), turmeric and/or curcuminoids (1-5%), black and or white pepper (1-5%), Garlic (1-5%), honey (2-10%), almond oil (0-10%) and salt (0-2%) with improved resistance to bacterial contamination compared to almond butter.

23) A composition of almond butter (60-98%), curcumin (1-8%), black and or white pepper (1-5%), Garlic (1-5%), honey (2-10%), almond oil (0-10%) and salt (0-2%) is a preferred embodiment.

24) A composition of almond butter (60-98%), curcuminoids and/or curcumin and/or turmeric (1-8%), a spice mix such as garam masala or other spice mix (1-8%), honey (2-10%), almond oil (0-10%) and optional salt (0-2%).

### EXAMPLES OF FORMULATIONS

### Formulation 1

Almonds dry roasted or raw or almond butter: 500 grams+71 grams if no almond oil is used. Black pepper: 16.4 grams, Salt 6.2 grams, Garlic 21.2 grams, Turmeric 41 grams, Honey 39.2 grams, Almond Oil 70.2 grams, cumin powder 12.4 grams, cinnamon powder 10.2 grams, marjoram powder 6.7 grams, chilly powder 6.9 grams, Ginger powder 25.6 grams.

Procedure: Grind the ingredients together in a grinding mill or other machine. One may stop at any stage of grinding to get a smooth, creamy, coarse or crunchy product. The spices may be in whole, powdered or paste form. Alternatively, a premade nut butter may be mixed with spice powders or whole spices and other ingredients and then ground/blended. The order of addition of ingredients is optional. Any of the ingredients or the final product may be heated (this is optional).

### Formulation 2

Almonds dry roasted or raw: 500 grams+71 grams if no almond oil is used

Black pepper: 16.4 grams, Salt 6.2 grams, Garlic 21.2 grams, Turmeric 41 grams, Honey 39.2 grams, Almond Oil 70.2 grams
Procedure: Grind the ingredients together in a grinding mill or other machine. One may stop at any stage of grinding to get a smooth, creamy, coarse or crunchy product. The spices may be in whole, powdered or paste form. Alternatively, a premade nut butter may be mixed with spice powders or whole spices and other ingredients and then ground/blended. The order of addition of ingredients is optional. Any of the ingredients or the final product may be heated (this is optional).

### Formulation 3

Pea nuts dry roasted or raw: 500 grams+71 grams if no oil is used.
Black pepper: 12 g, turmeric 12 g, garlic 20 g, ginger 20 g, salt 4 g, honey 20 g, peanut oil 12 g, cumin 12 g, cinnamon 10 g, marjoram 20 g, chillipowder 7 g,
Procedure: Grind the ingredients together in a grinding mill or other machine. One may stop at any stage of grinding to get a smooth, creamy, coarse or crunchy product. The spices may be in whole, powdered or paste form. Alternatively, a premade nut butter may be mixed with spice powders or whole spices and other ingredients and then ground/blended. The order of addition of ingredients is optional. Any of the ingredients or the final product may be heated (this is optional).

### Formulation 4

Almond Butter, (salted or unsalted, raw or roasted) 300.00 lb, Honey, 19.65 lb, Turmeric powder 7.22 lb, Black Pepper, ground 6.96 lb, Garlic Powder 5.12 lb, Salt 2.00 lb
Procedure: The starting nut butter may be smooth, creamy, coarse or crunchy. The spices may be in whole, powdered or paste form. The nut butter is mixed with spice powders or whole spices and other ingredients and then ground/blended. The order of addition of ingredients is optional. Any of the ingredients or the final product may be heated (this is optional).

### Formulation 5

### Ingredients

Almond butter (raw or roasted) 300.0 lb, Honey 19.65 lb, Almond oil 19 lb, Turmeric powder 7.22 lb, Black Pepper, ground 6.96 lb, Garlic Powder 5.12 lb, Salt 2.00 lb
Procedure: The starting nut butter may be smooth, creamy, coarse or crunchy. The spices may be in whole, powdered or paste form. The nut butter is mixed with spice powders or whole spices and other ingredients and then ground/blended. The order of addition of ingredients is optional. Any of the ingredients or the final product may be heated (this is optional).

### Formulation 6

### Ingredients

Almonds (raw or roasted) 300.0 lb, Honey 19.65 lb, Almond oil 19 lb, Turmeric powder 7.22 lb
Black Pepper, ground 6.96 lb, Garlic Powder 5.12 lb, Salt 2.00 lb
Procedure: The starting nut butter may be smooth, creamy, coarse or crunchy. The spices may be in whole, powdered or paste form. The nut butter is mixed with spice powders or whole spices and other ingredients and then ground/blended. The order of addition of ingredients is optional. Any of the ingredients or the final product may be heated (this is optional).

### Formulation 7

### Ingredients

Peanut butter (raw or roasted) 300.0 lb, Honey 19.65 lb, Turmeric powder 7.22 lb
Black Pepper, ground 6.96 lb, Garlic Powder 5.12 lb, Salt 2.00 lb
Procedure: The starting nut butter may be smooth, creamy, coarse or crunchy. The spices may be in whole, powdered or paste form. The nut butter is mixed with spice powders or whole spices and other ingredients and then ground/blended. The order of addition of ingredients is optional. Any of the ingredients or the final product may be heated (this is optional).

### Formulation 8

### Ingredients

Almond butter (raw or roasted) 100.0 lb, Peanut butter (raw or roasted) 200 lb
Maple syrup 19.65 lb, Olive oil 19 lb, curcuminoids 7 lb, Black Pepper, ground 6.96 lb
Garlic Powder 5.12 lb, potassium chloride 2.00 lb
Procedure: The starting nut butter may be smooth, creamy, coarse or crunchy. The spices may be in whole, powdered or paste form. The nut butter is mixed with spice powders or whole spices and other ingredients and then ground/blended. The order of addition of ingredients is optional. Any of the ingredients or the final product may be heated (this is optional).

### Formulation 9

### Ingredients

Almonds (raw or roasted) 160.0 lb, Peanuts (raw or roasted) 170 lb, Honey 30 lb
Almond oil 10 lb, peanut oil 10 lb, Turmeric 7.22 lb, Black Pepper, ground 6.96 lb

### Garlic Paste 5.12 lb, Salt 2.00 lb

Procedure: The nuts are ground in a grinding mill along with the other ingredients and spices and mixed thoroughly. The product may be heated if desired but heating is optional. The final product may be smooth, creamy, coarse or crunchy. The order of addition of ingredients is optional. The nuts may be ground separately or together. The other ingredients may be added initially during the nut grinding or later. The spices may be in powdered or paste form or one may use whole spices and grind them either during the grinding/blending or before.

### Formulation 10

Pea nuts dry roasted or raw: 200 g, Turmeric 6 grams
Black pepper: 4 grams, Salt: 1 gram, Garlic 4 gram, Ginger powder 4 grams
Molasses 4 grams, Almond Oil 18 grams, cumin powder 2 grams, cinnamon powder 2 grams marjoram powder 3 grams, chilly powder 1 gram
The nuts are ground in a grinding mill along with the other ingredients and spices and mixed thoroughly. The product may be heated if desired but heating is optional. The final product may be smooth, creamy, coarse or crunchy. The order of addition of ingredients is optional. The other ingredients may be added initially during the nut grinding or later. The spices may be in powdered or paste form or one may use whole spices and grind them either during the grinding/blending or before.

### Formulation 11

Cashew nut butter dry roasted or raw (or cashew nuts if grinding is done with the other ingredients) 500 grams
Turmeric 6 grams, Black pepper: 4 grams, Salt: 1 gram, Ginger powder 4 grams
Fruit concentrate 4 grams, cumin powder 2 grams, vitamin B 2 g, wheat germ 30 grams, egg white 3 g, vinegar 10 ml.
Procedure: The nuts are ground in a grinding mill or other grinding machine along with the other ingredients and spices and mixed thoroughly. The product may be heated if desired but heating is optional. The final product may be smooth, creamy, coarse or crunchy. The order of addition of ingredients is optional. Alternatively, one may grind the nuts first and then add the other ingredients and blend them. Another optional method is to use a premade nut butter and blend in the other ingredients with or without heating.

### Formulation 12

Sesame seeds: 500 grams, Turmeric 6 grams, curcuminoids 2 g, Black pepper: 4 grams, Salt: 1 gram, Garlic 4 gram, Ginger powder 4 grams, Honey 4 grams, Canola Oil 18 grams, cumin powder 2 grams, cinnamon powder 2 grams, marjoram powder 3 grams, chilli powder 1 gram.
Procedure: The sesame seeds are ground in a grinding mill/other grinder along with the other ingredients and spices (in powdered form) and mixed thoroughly. Alternatively, one may start from sesame tahini. The product may be heated if desired but heating is optional. The final product may be smooth, creamy, coarse or crunchy. The order of addition of ingredients is optional.

### Formulation 13

Peanut butter 250 grams, walnut butter 150 grams, almond butter 100 grams (or nuts if the procedure involves grinding the nuts in a mill), curcumin 6 grams, Black pepper: 4 grams, Agave nectar 20 grams, Salt substitute 1 gram
Procedure: The nuts butters are mixed with the other ingredients and spices and mixed thoroughly. The product may be heated if desired but heating is optional. The starting nut butters and final product may be smooth, creamy, coarse or crunchy. The order of addition of ingredients is optional. The nuts may be ground separately or together. The spices and curcumin may be in powdered or paste form or one may use whole spices and grind them either during the grinding/blending or before.

### Formulation 14

Walnuts dry roasted or raw: 500 grams+71 grams if no almond oil is used
Black pepper powder: 16 g, Salt 4 g, Garlic 20 g, Ginger powder 24 g, Turmeric 12 g, mollasses 40 g, Almond Oil 72 g, cumin powder 12 g, cinnamon powder 10 g, marjoram powder 20 g, chilli powder 7 g.
Procedure: The nuts are ground in a grinding mill along with the other ingredients and spices and mixed thoroughly. The product may be heated if desired but heating is optional. The final product may be smooth, creamy, coarse or crunchy. The order of addition of ingredients is optional. The other ingredients may be added initially during the nut grinding or later. The spices may be in powdered or paste form or one may use whole spices and grind them either during the grinding/blending or before. Alternatively, one may use a premade nut butter.

### Formulation 15

Cashew butter, 50 g, Peanut butter 46 g, turmeric: 4 g
Procedure: If whole turmeric is used, it is broken down by milling/grinding and blended with the nut butters. If turmeric powder is used, it can be blended with the nut butters. The product may be heated if desired but heating is optional. The final product may be smooth, creamy, coarse or crunchy. The order of addition of ingredients is optional. Alternatively, one may use a premade nut butter.

### Formulation 16

Almond butter 85 g, Raisins 7 g, garbanzo bean powder, green tea extract 2 g, turmeric 2 g, pomegranate concentrate 3 g.

Procedure: The ingredients are mixed and blended. The order of addition is optional. Heating of the ingredients or products is optional. Any of the ingredients may be roasted before adding.

### Formulation 17

Almond butter 80 g, turmeric 2 g, curcumin 2 g, black pepper 1 g, garlic 1 g, almond oil 7 g (optional), honey 7 g, salt 0.5 g.

Procedure: The ingredients are mixed and blended. The order of addition is optional. Heating of the ingredients or products is optional.

### Formulation 18

Almond butter 127 g, Curcumin 2.6 g
Procedure: The ingredients are mixed and blended. The order of addition is optional. Heating of the ingredients or products is optional.

### Formulation 19

Almond butter 127 g, turmeric 3.6 g
Procedure: The ingredients are mixed and blended. The order of addition is optional. Heating of the ingredients or products is optional.

The above examples representations of possible formulations and does not limit the patent in any way.

The processes used for the manufacture of P include but is not limited to the example below.
1) Blending curmin/curcuminoids/turmeric with or without other spices and/or flavors in minimum amount of oil (oil is optional)
2) Blending the same under shear to create a uniform suspension/mixture
3) Optional heating for extracting flavor and/or pasteurization
4) Optional pasteurization of sweetening agents such as honey
5) Blending of the curcumin/turmeric/curcuminoids—spice-flavor suspension with the sweetener.
6) Addition of nut/seed butter and homogenization with optional heating.
7) The order of addition/mixing of ingredients is variable.

Addition of nuts/seeds, spices, sweetening agents, additives and flavors into a nut/seed grinding machine followed by grinding of all components is an alternative method. The order of addition of ingredients in any of these methods is variable. For example, one may add the nuts first and then add the spices. Or, one may add all the ingredients in a nutbutter grinding mill/chopping machine or other machine and make the spread. One may start from the nuts and/or seeds or nut/seed butter or a mixture of nut and seed butters.

Combination of nut/seed butters to cu/co/tu/mx, other spices, sweeteners, flavors, preservatives or other additives may be done in any order. One or more of the ingredients may be heated. The spices used may be a premade spice mix such as garam masala or one may start from individual spices or spice powders or spice pastes or mixtures thereof.

The two preferred methods for producing nut/seed butters containing cu/co/tc/tu/mx with improved oxidative stability, texture, flavor and improves resistance to bacterial growth described above are representative examples. The methods for the production of P are not limited to these processes.

The water activity of the product is in the range 0.3-0.6. The most preferred composition was sampled by a team of experts. The unanimous conclusion was that the taste and flavor of the spread produced was considerably improved compared to that of the starting nut butter. Sampling was done after one day of production and the flavor had improved further. This could be because of the extraction of spices and flavors in the nut butter medium.

Oil separation Experiment: Samples of two embodiments of P and control (Parent almond butter) were stirred and poured into 16 OZ jars of the same size. After allowing for settling, the weight of the oil separated was measured. Average of three measurements were used for calculation. The formulations both exhibited significantly reduced oil separation compared to the parent nut butter (53% and 64% respectively)

| | All natural Almond | Turmeric Almond | Turmeric Almond |
|---|---|---|---|
| | Butter (control) | Spread P | Spread P |
| | Amount of the | embodiment P1 | embodiment P2 |
| Experiment | supernatant | Amount of the | Amount of the |
| Number | oil (g) | supernatant oil (g) | supernatant oil (g) |
| 1 | 21 | 11 g | 8 |
| 2 | 22 | 10 g | 7 |
| 3 | 21 | 9 | 8 |
| Average | 21.3 | 10 g | 7.67 |
| Percentage | NA | 53% | 64% |
| Reduction | | | |

The measurements were made with three 1 lb jars of each type.
Embodiment P1 contained
Almond butter 90%, Sugar 2%, Turmeric 6%, Black pepper 0.5%,
Garlic .25%, Salt 0.25%.
Embodiment P2 contained
Almond butter 80%, Honey 4%, Turmeric + curcumin 8%, Garlic 3%,
Black pepper 4%, Salt 1%.
$Percentage Reduction = 100 * \frac{\begin{matrix}Oil separation in Control nut butter - \\ Oil Separation in the embodiment\end{matrix}}{\begin{matrix}Oil separation in control nut butter \\ \end{matrix}}$

The antioxidant potential of the product P was measured for one embodiment P3 (almond butter 84%, Honey 5%, Almond oil 5%, turmeric 2.5%, black pepper 2% garlic 1%, salt 0.5%) and a control. The values for antioxidant potential for a) Plain almond butter (b) an embodiment containing P3 2.5% turmeric were measured by the Oxygen Radical Absorbing Capacity (ORAC) assay [14] for antioxidant capacities.

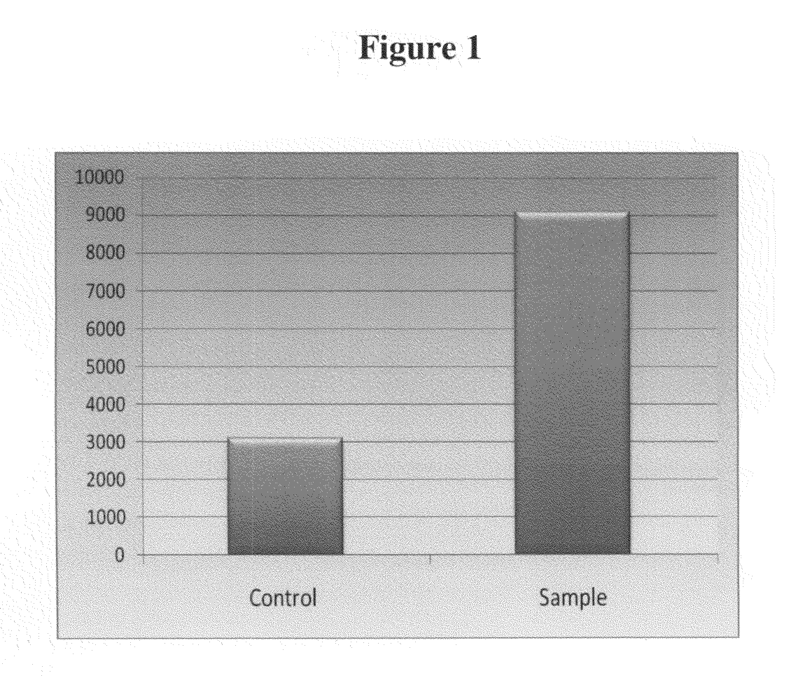

A major component of nut butters such as almond butter is the nut oil. Oils are glycerol esters of long chain fatty acids. If the fatty acid is unsaturated, that is if it has double bonds, the oil is considered an unsaturated oil. Mono unsaturated and poly unsaturated oils and fats contain one or more double bonds per alkyl chain. These double bonds are susceptible to oxidation by atmospheric oxygen and this results in the oil becoming rancid. Because of this, all natural nut butters have a limited shelf life. Current methods of extending shelf life include addition of chemical preservatives, the effect of these on the health is not clearly demonstrated. Many health conscious consumers prefer not to consume foods containing such artificial preservatives. From FIG. 1, it is clear that the composition P3 has nearly three times the ORAC value as the parent nut butter. This implies that its resistance to oxidative damage is increased 300%. Here, turmeric serves as an all natural, beneficial additive that reduces the oxidative damage. This will result in slowing down the process of rancidification thus extending the shelf life of the nut butter.

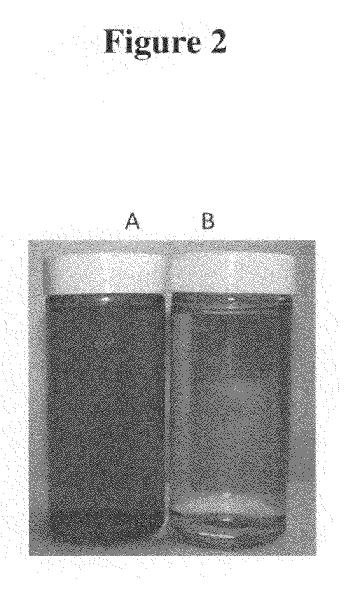

Another advantage of this formulation over plain cu/co/tc/tu/mx is the improved solubility. cu/co/tc/tu/mx has very low solubility in water and this is likely to be one of the major reasons of the very low bioabsorption of curcumin. The solubility of curcumin in a nut butter medium is very high. We performed the following experiment to compare the solubilities of curcumin in water and almond oil. (Almond oil is a major component of almond butter and was chosen as a model to establish the curcumin solubility in the nut butter because it is transparent). We added 1 g of curcumin to 100 ml almond oil, stirred and filtered to a vial. The filtrate was yellow in color. A control experiment was performed with water and the filtrate was found to be colorless. A photograph of these vials is shown in FIG. 2. From the color which arises from curcumin (−425 nm absorption maximum) it is clear that it is very soluble in the almond oil, but not soluble in water.

Curcuminoids refers to [(1E,6E)-1,7-bis(4-hydroxy-3-methoxyphenyl) hepta-1,6-diene 3,5-dione]. Curcumin is the primary active ingredient of turmeric. In addition to curcumin, turmeric also contains other active molecules. The most important active molecules in turmeric, including curcumin are collectively known as curcuminoids. Curcuminoids include curcumin, demethoxycurcumin and bisdemethoxycurcumin. Nut/seed butters refer to the substance obtained by grinding, chopping, milling or other break down of nuts/seeds irrespective of the process used. The nut/seed butter includes these in smooth, creamy, coarse or crunchy form. The nut/butter may be produced by any breakdown method such as grinding, milling, chopping etc or a combination of these.

Nuts/seeds/powdered nuts//nut butters/seed butters/powdered seeds/mixtures of these are represented as nu/se/pn/nb/sb/ps/mns. Nuts, powdered nuts and nut butters in nu/se/pn/nb/sb/ps/mns include but are not limited to almonds, peanuts, pistachios, cashews, macademia, pine, walnut, hazelnut, chestnut, brazilnut, soynut, filberts, hickory, pecans in ground, powdered, crunchy, smooth, paste, whole, crushed, broken or slurry form. The seeds and seed butters include but are not limited to the following: sesame seeds, sunflower seeds or other seeds in ground, powdered, crunchy, smooth, paste, whole, crushed, broken or slurry form. Either one of the nuts or seeds or a combination of two or more types of nuts or seeds or mixtures of nuts and seeds may be used.

Cu refers to curcumin, co refers to curcuminoids, tu refers to turmeric, tc refers to compounds extracted from turmeric, mix refers to a mixture containing one or more of cu, co, tc or tu.

Examples of spices mixed along with cu/co/tc/tu/mx include but are not limited to garlic, black pepper, white pepper, pepper corns, clove, cumin, nutmeg, mace, thyme, cinnamon, coriander, oregano, parsley, basil, holy basil, allspice, cilantro, rosemary, aniseed, fennel, dill (seed, herb or weed), cardamom, paprika, saffron, marjoram, mint, fenugreek, ginger, star anise, pink pepper, sage, or other spice, powdered, whole or as a paste or mixtures thereof. In addition spices may be selected from the following or mixtures thereof, these may be used in less preferred embodiments: Ajwain, Akudjura, alexanders, alkanet, allspice, galangal, amchur (mango powder), anise, aniseed myrtle, annato, apple mint, azafoetida, avocado leaf, barberry, bay leaf, borage, black cardamom, black mustard, blue fenugreek, brown mustard, calamint, calendula, candlenut, capers, caraway, catnip, cassia, cayenne pepper, celery seed, chamomile, chervil, chicory, chili pepper, chilli powder, chipotle, chives, cicely, cilantro, cinnamon myrtle, clary, clary sage, cost mary, cuban oregano, cubeb pepper, cud weed, culantro, curry leaf, curry plant, dorrigo pepper, elder flower, epazote, culantro (long coriander), fingerroot, french sorel, galingale, garlic chives, ginkgo nuts, golpar, grains of paradise, grains of selim, green tea, ground ivy, hops, horseradish, houttuynia, hot pepper, hyssop, jasmine, juniper berry, jalepenos, kaffir lime, kaffir lime leaves, lokam, lavender, laser (silphium), lemon balm, lemon basil, lemon grass, lemon mint, lemon myrtle, lemon thyme, lemon verbena, lesser galangal, licorice, lime flower, linden flower, rice paddy herb, long pepper, lovage, luohanguo, mace, marjoram, nasturtium, nigella, black caraway, olida, orris root, sweet cicely, pandan flower (kewra), pandan leaf, paprika, paracress, peppermint, peppermint gum, perilla, piper sarmentosum, white pumpkin, poppy seed, ramsons (wood garlic), red pepper, ribbery, rue, safflower, saigon cinnamon, salad burnet, salep, sassafras, savory, sesame seed, sheep's sorrel, sichuan pepper, sloe berries, sorrel, spearmint, spikenard, sumac, summer savory, sweet woodruff, talinum, tamarind, tansy, tarragon, tasmanian pepper, tea, felty germander, that basil, tulsi, vanilla, vietnameese balm, vietnamese cinnamon, vietnamese corriander, wasabi, water-pepper, water cress, wattle seed, white mustard, wild thyme, winter savory, wintergreen, wood avens, woodruff, wormwood, yellow mustard, yerba buena, zaatar herb, zedoary, lemon, celery, celery seed, nigella seed, poppy seed, pimenta, pimento, orange peel, rapeseed, nori, mustard seeds, pimento, scallions, sweet pepper. Spice mixtures including but not limited to berbere, curry powder, chinese five spice powder, garam masala, chaat masala, goda masala, Herbes de Provence, kaala masala, Khmeli suneli, pudding spice, panch phoron, pumpkin pie spice, quatre epices, rasel hanout, tandoori masala, zaatar, jerk spice.

The spices may be added individually or as a premade mix such as Garam masala or other spice mix.

The entire contents including the references cited therein of the patents and publications mentioned in this specification are incorporated by reference in their entirety for all purposes to the same extent as if each individual patent, patent application or publication were so individually denoted.
1) Goel, Ajay; Jhurani, Sonia; Aggarwal, Bharat B. “Multi-targeted therapy by curcumin: how spicy is it?” Molecular Nutrition & Food Research (2008), 52(9), 1010-1030.
2) Campbell, Frederick C.; Collett, Gavin P. “Chemopreventive properties of curcumin.” Future Oncology (2005), 1(3), 405-414
3) (a) Raja, K. S.*, Balambika, R*., Sukanta Dolai, Wei Shi “The Concept of a Green Drug, Curcumin and it's derivatives as a model system.” Mini Reviews in Organic Chemistry, (2009), Volume 6, No. 2. 152-158. (b) Shi, W.; Dolai, S.; Rizk, S.; Hussain, A.; Tariq, H.; Averick, S.; L′Amoreaux, W.; El Idrissi, A.; Banerjee, P.; Raja, K. S. Org. Lett., 2007, 9, 5461.
4) Strimpakos A. S, Sharma R. A. “Curcumin: preventive and therapeutic properties in laboratory studies and clinical trials.” Antioxid Redox Signal. 2008 10(3):511-45.
5). Braga, Mara E. M.; Leal, Patricia F.; Carvalho, Joao E.; Meireles, M. Angela A. “Comparison of yield, composition, and antioxidant activity of turmeric (Curcuma longa L.) extracts obtained using various techniques.” Journal of Agricultural and Food Chemistry (2003), 51(22), 6604-6611
6) Yeh, C. H, Chen, T. P, Wu, Y. C, Lin, Y. M, Jing, L. P. “Inhibition of NF□ B Activation with Curcumin Attenuates Plasma Inflammatory Cytokines Surge and Cardiomyocytic Apoptosis Following Cardiac Ischemia/Reperfusion.” Journal of Surgical Research (2005), 125(1), 109-116.
7) Cole G. M, Teter B, and Frautschy S. A. “Neuroprotective effects of curcumin.” Adv Exp Med. Biol. (2007); 595:197-212.
8) Rossi, L.; Mazzitelli, S.; Arciello, M.; Capo, C. R.; Rotilio, G. “Neurochemical Benefits from Dietary Polyphenols for Brain Aging and Alzheimer's Disease.” Research (2008), 33(12), 2390-2400.
9) Park, Byeoung-Soo; Kim, Jae-Gyu; Kim, Mi-Ran; Lee, Sung-Eun; Takeoka, Gary R.; Oh, Ki-Bong; Kim, Jeong-Han. “Curcuma longa L. Constituents Inhibit Sortase A and Staphylococcus aureus Cell Adhesion to Fibronectin.” Journal of Agricultural and Food Chemistry (2005), 53(23), 9005-9009.
10) Jain, T.; Ashish; J. Devasagayam, T. P. A. “Cardioprotective and other beneficial effects of some Indian medicinal plants.” Journal of Clinical Biochemistry and Nutrition (2006), 38(1), 9-18.
11) Hong-Liang Li, Chen Liu, Geoffrey de Couto, Maral Ouzounian, Mei Sun, 1 Ai-Bing Wang, Yue Huang, Cheng-Wei He, Yu Shi, 1 Xin Chen, Mai P. Nghiem, Youan Liu, Manyin Chen, Fayez Dawood, Masahiro Fukuoka, Yuichiro Maekawa, Liyong Zhang, Andrew Leask, Asish K. Ghosh, Lorrie A. Kirshenbaum, and Peter P. Liu, “Curcumin prevents and reverses murinecardiac hypertrophy” The Journal of Clinical Investigation (2008) Volume 118 Number 3.
12) Mohandas, K. M., Desai, D. C., “Epidemiology of digestive tract cancers in India. V. Large and small bowel. Indian Journal of Gastroenterology” (1999) 18 (3), 118-121.
13) Cheng, A. L.; Hsu, C. H.; Lin, J. K.; Hsu, M. M.; Ho, Y. F.; Shen, T. S.; Ko, J. Y.; Lin, J. T.; Lin, B. R.; Wu, M. S.; Yu, H. S.; Jee, S. H.; Chen, G. S.; Chen, T. M.; Chen, C. A.; Lai, M. K.; Pu, Y. S.; Pan, M. H.; Wang, Y. J.; Tsai, C. C.; Hsieh, C. Y. “Phase I clinical trial of curcumin, a chemopreventive agent, in patients with high-risk or pre-malignant lesions.” Anticancer Res. (2001), 21, 2895.
14) Prior, R. L.; Hoang, H.; Gu, L.; Wu, X.; Bacchiocca, M.; Howard, L.; Hampsch-Woodill, M.; Huang, D.; Ou, B.; Jacob, R. “Assays for hydrophilic and lipophilic antioxidant capacity (oxygen radical absorbance capacity (ORACFL)) of plasma and other biological and food samples.” J. Agric. Food Chem. (2003), 51, 3273-3279

Although the present invention has been described with reference to specific details of certain embodiments thereof, it is not intended that such detail should be regarded as limitations upon the scope of the invention, except as and to the extent that they are included in the accompanying claims.

What is claimed is:

1. A turmeric, curcumin, turmeric component or curcuminoid modified nut or seed butter spread which is resistant to oxidation, antibacterial and possessing human health benefits comprising: 0.2-70% of components selected from the group consisting of curcumin, curcuminoids, turmeric components, and turmeric; and 30-99.8% of a dispersing medium derived from nut or seed particles or nut butters wherein said nuts or seed are selected from the group consisting of almonds, peanuts, pistachios, cashews, macademia, pine, walnut, hazelnut, chestnut, brazilnut, soynut, filberts, hickory, pecans, sesame seeds, sunflower seeds or other seeds and mixtures thereof.

2. The nut or seed butter spread of claim 1 further comprising spices selected from the group consisting of garlic, black pepper, white pepper, pepper corns, clove, cumin, nutmeg, mace, thyme, cinnamon, coriander, oregano, parsley, basil, holy basil, allspice, cilantro, rosemary, aniseed, fennel, dill (seed, herb or weed), cardamom, paprika, saffron, marjoram, mint, fenugreek, ginger, star anise, pink pepper, sage, or other spice, Ajwain, Akudjura, alexanders, alkanet, allspice, galangal, amchur (mango powder), anise, aniseed myrtle, annato, apple mint, azafoetida, avocado leaf, barberry, bay leaf, borage, black cardamom, black mustard, blue fenugreek, brown mustard, calamint, calendula, candlenut, capers, caraway, catnip, cassia, cayenne pepper, celery seed, chamomile, chervil, chicory, chili pepper, chilli powder, chipotle, chives, cicely, cilantro, cinnamon myrtle, clary, clary sage, cost mary, cuban oregano, cubeb pepper, cud weed, culantro, curry leaf, curry plant, dorrigo pepper, elder flower, epazote, culantro (long coriander), fingerroot, french sorel, galingale, garlic chives, ginkgo nuts, golpar, grains of paradise, grains of selim, green tea, ground ivy, hops, horseradish, houttuynia, hot pepper, hyssop, jasmine, juniper berry, jalepenos, kaffir lime, kaffir lime leaves, lokam, lavender, laser (silphium), lemon balm, lemon basil, lemon grass, lemon mint, lemon myrtle, lemon thyme, lemon verbena, lesser galangal, licorice, lime flower, linden flower, rice paddy herb, long pepper, lovage, luohanguo, mace, marjoram, nasturtium, nigella, black caraway, olida, orris root, sweet cicely, pandan flower (kewra), pandan leaf, paprika, paracress, peppermint, peppermint gum, perilla, piper sarmentosum, white pumpkin, poppy seed, ramsons (wood garlic), red pepper, ribbery, rue, safflower, saigon cinnamon, salad burnet, salep, sassafras, savory, sesame seed, sheep's sorrel, sichuan pepper, sloe berries, sorrel, spearmint, spikenard, sumac, summer savory, sweet woodruff, talinum, tamarind, tansy, tarragon, tasmanian pepper, tea, felty germander, that basil, tulsi, vanilla, vietnameese balm, vietnamese cinnamon, vietnamese corriander, wasabi, water-pepper, water cress, wattle seed, white mustard, wild thyme, winter savory, wintergreen, wood avens, woodruff, wormwood, yellow mustard, yerba buena, zaatar herb, zedoary, lemon, celery, celery seed, nigella seed, poppy seed, pimenta, pimento, orange peel, rapeseed, nori, mustard seeds, pimento, scallions, sweet pepper. Spice mixtures including but not limited to berbere, curry powder, chinese five spice powder, garam masala, chaat masala, goda masala, Herbes de Provence, kaala masala, Khmeli suneli, pudding spice, panch phoron, pumpkin pie spice, quatre epices, rasel hanout, tandoori masala, zaatar, jerk spice and mixtures thereof.

3. The nut or seed butter spread of claim 2 further comprising sweeteners selected from the group consisting of: honey, sugar including cane sugar, beet sugar, barley sugar brown sugar, raw sugar including turbinado sugar, jaggery, muscovedo, panela, sucanat, palm sugar, amazake, caramel, molasses, maple syrup, taffy, agave nectar, corn syrup, birch syrup, pine syrup, hickory syrup, poplar syrup, palm syrup, sugar beet syrup, sorghum syrup, cane syrup, cane sugar, golden syrup, barley malt syrup, rice syrup, agave syrup, yacon syrup, fruit powder or paste, fruit juice, fruit and vegetable sugars including pumpkin sugar, watermelon sugar, date sugar, pekmez, vegetables or vegetable extract including carrots, beets, celery sugar cane juice, carbohydrates including glucose, dextrose, fructose, levulose, invert sugar/invert syrup, galactose, sucrose, lactose or mixtures thereof, stevia, Jiaogulan, sugar substitutes including acesulfame potassium, salt of aspartame-acesulfame, acesulfame, alitame, aspartame, anethole, cyclamate, glycyrrhizin, lo han guo, neotame, perillartine, saccharin, stevioside, sucralose, inulin, sugar alcohols including glycol, glycerol, erythritol, threitol, arabitol, xylitol, ribitol, mannitol, sorbitol, dulcitol, iditol, isomalt, maltitol, lactitol, polyglycitol, natural sweeteners including brazzein, curculin, hydrogenated starch hydrolysates, mabinlin, miraculin, monatin, monellin, pentadin, tagatose, thaumatin and mixtures thereof.

4. The nut or seed butter spread of claim 3 further comprising ingredients selected from the group consisting of salts and salt substitutes selected from the group consisting of salt (NaCl), sea salt, rock salt, potassium chloride, potassium lactate, black salt, pink salt, smoked salt or other salt or salt substitute and mixtures thereof.

5. The nut or seed butter spread of claim 4 further comprising oils selected from the group consisting of peanut oil, walnut oil, almond oil, cotton seed oil, olive oil, corn oil, canola oil, ghee, butter, margarine, coconut oil, saffola oil, sunflower oil, mustard oil, cashew oil, palm oil, soybean oil, rapeseed oil, hazelnut oil, pecan oil, pine nut oil, other nut oils, linseed oils, rice bran oil, fish oil, margarine, shortening, partially or fully hydrogenated oils or fats, mono and diglycerides, hazelnut oil, macademia nut oil, mongongo nut oil (manketti oil), pine nut oil, pistachio oil, pumpkin seed, watermelon, sesame or other seed oils, apricot oil, amaranth oil, apple seed oil, argan oil, artichoke oil, avocado oil, babassu oil, ben oil borneo tallow nut oil, cape chestnut oil, cocoa butter, carob pod oil, coriander seed oil, dika oil, flax oil, false flax seed oil, grape seed oil, kapok seed oil, pine nut oil, lard, lallemantia oil, jojoba oil, marula oil, meadofoam seed oil, nutmeg butter, okra seed oil, papaya seed oil, preilla seed oil, pequi oil, poppy seed oil, primrose oil, prune kernel oil, quinoa oil, ramtil oil, royle oil, sacha-inchi oil, soy oil, tea seed oil, thisle oil, tomato seed oil wheat germ oil, mono or di glycerides, or other oils, fats or oil substitutes and mixtures thereof.

6. The nut or seed butter spread of claim 5 further comprising acidulants and acidity modifiers selected from the group consisting of citric acid, vinegar, lemon juice, dilute acetic acid, tartaric acid, lactic acid, malic acid, lime juice, sodium bicarbonate, sodium carbonate or the salts of the above acids and mixtures thereof.

7. The nut or seed butter spread of claim 6 further comprising grains, pulses, tubers or starches selected from the group consisting of wheat, wheat germ, broken wheat, rice, corn, potato, barley, maize, sorghum, oats, millets, rye, triticale, buckwheat, bread crumbs, corn crumbs, corn starch, katakuri starch, sago, tapioca, potato, arrow root or other tubers, other starches, fonio, quinoa, spelt, amaranth, kaniwa, durum, semolina, pulses including but not limited to kidney beans, black beans, mung bean, black gram, dal, garbanzo beans, lima bean, fava bean, lentils, peas including but not limited to green pea, yellow pea, cow pea, or other grains, tubers, peas or beans and mixtures thereof.

8. The nut or seed butter spread of claim 7 further comprising other additives selected from the group consisting of fruits, vegetables, eggs, milk products, vitamins, amino acids, minerals, herbs, food additives, texture additives, flavors, preservatives, and colors and wherein said additives are selected from the group consisting of raisins, prunes, apples, apricots, berries, amla (Indian gooseberry), jamoon, dates, citron, figs, cranberries or other fruit or vegetables including but not limited to carrots, or other vegetables in ground, juice, grated or other form, eggs, egg yolks, egg whites or egg substitutes, cheese, whey, milk solids, yoghurt or other milk substitute, vitamins, amino acids, proteins, minerals or herbs (culinary or medicinal, including but not limited to chamomile, psyllium, brahmi, pippali [long pepper], bael, tea (including but not limited to green, black) aloe, satavari, asparagus, eucalyptus, sandalwood, saw palmetto, neem) plants or extracts of herbs or plants (including but not limited to reserveratrol, green tea extract, bromelain, pomegranate extract), vegemite, wheat gluten, gluten, glycerol mono stearate, yeast, yeast extract, camphor, food additives (including but not limited to silicon dioxide, silicates, stearic acid, tartaric acid; emulsifiers including but not limited to lecithin, polysorbate 20, flavor enhancers including but not limited to MSG, glutamic acid, inosinic acid, maltitol; humectants including but not limited to urea, quillaaia, desiccants, stabilizers including but not limited to agar, pectin, texture additives including but not limited to collagen, gelatin carrageenan, alginin, guar gum, locust bean gum, xanthan gum, flavors, chocolate, cocoa, preservatives including but not limited to sodium benzoate, benzoic acid, calcium propionate, potassium sorbate, nitrates, nitrites, sulfites, bisulfites, disodium EDTA, BHA, BHT or colors including but not limited to FD&C blue no 1, 2, green 3, red 3, 40, yellow 5, 6.

9. The nut or seed butter spread of claim 1 having at least a 20% improvement in oxidative stability.

10. The nut or seed butter spread of claim 1 having improved resistance to bacterial growth compared to the parent nut or seed butter.

11. The nut or seed butter spread of claim 1 having at a least 20% improved resistance to oil separation.

12. The nut or seed butter spread of claim 1 having at least two times improved solubility of turmeric/curcumin/curcuminoids compared to a hydrophilic medium such as water.

13. The nut or seed butter spread of claim 1 wherein the nut/seed butter is selected from a group of peanut, pistachio, walnut, cashew, almond, soynut, brazilnut, pinenut, hazelnut, macademia nut, chestnut, pecans, filberts, hickory, sesame seed, sunflower seed and mixtures thereof.

14. The nut or seed butter spread of claim 1 made by a process whereby whole nuts are blended with turmeric/curcumin/curcuminoids and broken down in a nut butter mill or other grinding, milling or pulverizing machine.

15. The nut or seed butter spread of claim 1 wherein the nut/seed butter is almond butter.

16. The nut or seed butter spread of claim 1 wherein the nut/seed butter is peanut butter.

17. The nut or seed butter spread of claim 1 wherein the curcumin, curcuminoids, turmeric components and turmeric is used in concentrations from 1-9%.

18. The nut or seed butter spread of claim 3 wherein the sweetener is honey in concentrations from 2-10%.

19. The nut or seed butter spread of claim 2 wherein the spices are selected from the group of pepper and garlic.

20. The nut or seed butter spread of claim 4 wherein the salt is common table salt, sodium chloride.

21. The nut or seed butter spread of claim 5 wherein the oil used is selected from the group consisting of almond and peanut oil.

22. An Almond butter/ground almond and cu/co/tu/mx spread composition comprising: 80-96% almond butter; 1-5% of components selected from the group consisting of curcumin, curcuminoids, turmeric, and turmeric components; 0-5% Black or white pepper; 0-5% Garlic; 0-10% Honey; 0-10% Almond oil and 0-2% salt.

23. A curcumin or curcuminoid modified nut or seed butter spread which is resistant to oxidation and possessing human health benefits comprising: 0.2-70% of components selected from the group of consisting of curcumin and curcuminoids; and 30-99.8% of nut or seed particles as a dispersing medium.
